(19) United States
(12) Patent Application Publication
(10) Pub. No.: US 2023/0158283 A1
(43) Pub. Date: May 25, 2023

(54) SYSTEMS AND METHODS FOR A SMART BANDAGE FOR MONITORING AND TREATING WOUNDS

(71) Applicant: California Institute of Technology, Pasadena, CA (US)

(72) Inventors: WEI GAO, Pasadena, CA (US); EHSAN SHIRZAEI SANI, Pasadena, CA (US)

(73) Assignee: California Institute of Technology, Pasadena, CA (US)

(21) Appl. No.: 17/993,743

(22) Filed: Nov. 23, 2022

Related U.S. Application Data
(60) Provisional application No. 63/282,610, filed on Nov. 23, 2021.

(51) Int. Cl. A61M 35/00; A61N 1/36; A61F 13/00

(52) U.S. Cl. CPC A61M 35/10; A61N 1/36031; A61F 13/00063; A61M 2230/20; A61M 2205/3584; A61M 2230/201; A61M 2205/0205; A61M 2230/208; A61F 2013/00285

(57) ABSTRACT

Systems and methods for a smart bandage for monitoring and treating wounds are provided herein. The smart bandage may be a smart, wearable, flexible multi-layer substrate that may include a system that can monitor wound characteristics, perform autonomous bioanalysis of wound characteristics to determine wound treatment plans in a non-invasive method, perform drug delivery or antimicrobial agent release to treat and prevent infections, and promote healing by electrical stimulation. The smart bandage may be equipped with a wireless network that can communicate with users, such as patients and medical providers, directly about a patient's wound condition and provide for on-demand wound treatment.

### RELATED APPLICATION

This application claims the benefit of U.S. Provisional Application No. 63/282,610 filed on Nov. 23, 2021, the contents of which are incorporated herein by reference in their entirety.

### TECHNICAL FIELD

The present disclosure relates generally to systems and methods for a smart bandage for monitoring and treating wounds. In particular, some implementations may relate to a flexible multi-layer substrate with a sensor disposed therein that can monitor characteristics of a wound and administer treatment to the wound, e.g., by releasing drugs and antimicrobial agents and/or electrical stimulation (ES).

### BACKGROUND

Wearable bioelectronic technology offers many advantages for personalized health monitoring. Wearable devices are non-invasive and present less user error than other monitoring methods. Additionally, wearable devices offer the potential to monitor health status over time as opposed to collecting a sample that reflects health status at only a snap shot in time. This type of real-time monitoring may offer more accurate and individualized diagnosis, treatment, and prevention for health conditions. Specifically, wearable devices can measure pulse, respiration rate, temperature, and other health status indicators.

Chronic non-healing wounds are one of the major and rapidly growing clinical complications all over the world. Current therapies frequently include emergent surgical interventions, while abuse and misapplication of therapeutic drugs can lead to an increased morbidity and mortality rate. Despite the urgent need for more effective, controllable, biocompatible, and easy-to-implement therapies with minimal side effects, conventional wound care devices have remained passive and cannot dynamically respond to variations in wounds' physiological microenvironment.

Further, existing wound treatment may be a manual process that requires assessing the wound, monitoring the wound's physiological characteristics, applying a bandage to the wound if needed, and treating the wound for infection. In addition, current wound care products are unable to provide information about the wound, such as status of the wound bed or wound healing rate over time. These steps are typically performed in a medical facility by a medical provider and generally require a patient to visit the facility to have general wound care and management conducted. However, there are many situations where receiving such treatment is difficult or impossible, for example, on the battlefield, in space, in developing or low or middle income countries, or during a global pandemic. Therefore, a system and method of dynamically treating wounds when outside of a medical facility, which allows for more constant, individualized care, monitoring, and treatment, was needed.

### SUMMARY

Systems and methods are described herein for a smart bandage for monitoring and treating wounds. In various embodiments, the smart bandage may be a fully-integrated wearable bioelectronic system that wirelessly and continuously monitors physiological conditions of the wound bed via a custom-developed multiplexed multimodal electrochemical biosensor array. The smart bandage may further perform non-invasive combination therapy through controlled anti-inflammatory/antimicrobial treatment and electrical stimulated tissue regeneration. In some embodiments, the smart bandage may be a wearable patch that is biocompatible, mechanically flexible, stretchable, and can conformally adhere to the skin/wound throughout portions of, or during the entire healing process of a wound. Various embodiments, may include a system for real-time metabolic and inflammatory monitoring that may allow for higher accuracy and electrochemical stability of the smart bandage for multiplexed spatial and temporal wound biomarker analysis. The combination of electrically modulated antimicrobial agent delivery and electrical stimulation in the wearable smart bandage may accelerate cutaneous chronic wound healing, as well as, overall wound healing in patients.

In certain embodiments, the smart bandage may be a wearable, flexible multi-layer substrate with multiplexed sensors disposed thereon that can monitor the physiological microenvironment of a wound and identify characteristics of the wound. The characteristics of the wound may be monitored via biosensors configured to detect metabolites, amino acids, bacteria, vitamins, minerals, hormones, antibodies, pH, UA level, ammonia level, lactate level, CRP level, glucose level, and other biomarkers. The smart bandage may include an antimicrobial reservoir or hydrogel within the flexible multi-substrate layer. The antimicrobial reservoir or hydrogel may be connected to an outlet, also disposed on the smart bandage, and adjacent to the skin of a patient so that antimicrobial agents and drugs may be released from the antimicrobial reservoir or hydrogel and dispensed from the outlet onto a patient's skin or into a patient's wound.

The smart bandage may further include an electrical stimulation module that can provide electrical stimulation to a patient to assist with wound healing and tissue regeneration. In embodiments, the smart bandage may also include a control module that obtains signals from sensors representative of wound characteristics and can perform a bioanalysis of the wound or transmit the signals wirelessly to a user, which may occur discretely or continuously. The control module can also receive signals for wound treatment from a user or autonomously and dynamically administer wound treatment based on programmed threshold parameters for particular treatments in reference to certain wound characteristics.

The smart bandage may further include a wireless communication module that can connect the smart bandage device to another wireless device, such as a mobile phone, computer, tablet, or medical station.

The control module on the smart bandage may include a non-transitory computer readable storage medium that includes instructions to receive and process signals from the sensors representative of wound characteristics, transmit the received signals via the wireless communication module to another wireless device, receive signals from a wireless device, transmit signals to the antimicrobial reservoir/hydrogel/outlet and electrical stimulation module, and in some embodiments, autonomously process sensor signals to determine whether they exceed threshold values and autonomously initiate treatment.

Wearable sensors integrated with telemedicine could support safe and efficient monitoring of individual health, which would allow for timely intervention for infection and treatment of wounds and other medical conditions.

Other features and aspects of the disclosure will become apparent from the following detailed description, taken in conjunction with the accompanying drawings, which illustrate, by way of example, the features in accordance with various embodiments. The summary is not intended to limit the scope of the invention, which is defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is an example box diagram depicting the smart bandage system, according to various embodiments of the disclosed technology.

FIG. 2 depicts an exploded example smart bandage device, according to various embodiments of the disclosed technology.

FIG. 3 is an example smart bandage application to an open wound, according to various embodiments of the disclosed technology.

FIG. 4 depicts an example application of a smart bandage device to a human foot, according to various embodiments of the disclosed technology.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

Wearable technology offers many advantages for personalized wound monitoring and treatment. Wearable devices are non-invasive and present less user error than other monitoring and treatment methods. Additionally, wearable devices offer the potential to monitor the health and recovery status of wounds over time as opposed to collecting a sample from a wound that reflects wound characteristics at only a discrete instance in time, i.e., when the sample was taken. Further, wearable devices may reduce the number of hospital visits for patients who would otherwise require regularized monitoring and treatment by a medical provider at an in person, patient facility. This type of real-time monitoring may offer more accurate and individualized treatment of wounds, as well as, continuous monitoring of wound characteristics and biomarkers. In embodiments, wearable devices can measure wound conditions and levels of infection to reduce the morbidity and infection rates of chronic wounds, and release treatments that increase healing of the wound.

Smart bandages are one type of wearable technologies that can be particularly desirable for chronic wound treatment because chronic wounds require regular medical care. Chronic wounds are characterized by impaired and stagnant healing, prolonged and uncontrollable inflammation, as well as compromised extracellular matrix (ECM) function. Over 6.7 million people in the United States suffer from chronic non-healing wounds including diabetic ulcers, non-healing surgical wounds, burns, and venous-related ulcerations, causing a staggering financial burden of over $25 billion per year on the healthcare system. In particular, chronic wounds may be caused by several pathologies including diabetes mellitus, vascular insufficiency, compromised nutritional and immunological states, surgeries, and burns.

Current and conventional wound therapies including skin grafts, skin substitutes, negative pressure wound therapy and others can be beneficial, but frequently require medical provider involvement, procedures, or surgical intervention. Additionally, these conventional therapies often fail to restore tissue homeostasis, and can lead to further health complications, such as microbial infection. Microbial infection at the wound site can prolong the healing process, and lead to necrosis, sepsis, and even death. Both topical and systemic antibiotics are increasingly prescribed to patients suffering from chronic non-healing wounds, but the overuse, abuse, and misapplication of antibiotics can lead to an escalating drug resistance in bacteria, causing increases in morbidity and mortality rates. As an alternative approach, electrical stimulation has shown to have a significant effect on the wound healing process, including stimulating fibroblast proliferation and differentiation into myofibroblasts and collagen formation, keratinocyte migration, angiogenesis, and attracting macrophages. However, conventional electrical stimulation devices require bulky equipment and wire connections, making them challenging for practical daily use. More effective, fully controllable, and easy-to-implement therapies, as described herein, are needed for personalized treatment of chronic wounds with minimal side effects.

Quantitative assessments of wound biomarkers and characteristics were investigated, and wearable wound management devices were constructed to monitor wound characteristics and autonomously or instructively provide treatments therein. Wearable devices may offer highly desirable, non-invasive, and continuous monitoring of various types of wounds, for example, chronic wounds. Wearable devices may provide an alternative to regular patient care by medical providers, which can include cleaning wounds, monitoring wound characteristics, applying antimicrobials, and active or passive wound diagnosis. In various embodiments, wearable wound management devices may be advantageous for improving chronic wound healing.

Chronic wound healing can be a complex biological process including four integrated and overlapping phases: hemostasis, inflammation, proliferation, and remodeling. At each stage of the healing process, the chemical composition of the wound exudate may change, this may indicate the stage of healing and even the presence of infection. For example, increased temperature may be associated with bacterial infection; acidity (pH) may indicate a healing state with balanced protease activities and effective ECM remodeling; elevated uric acid (UA) may indicate wound severity with excessive reactive oxygen and inflammation; lactate and ammonium may be biomarkers for soft-tissue infection diagnosis in diabetic foot ulcers; and wound exudate glucose may have a correlation with blood glucose and bacterial activities, which may provide therapeutic guidance for clinical diabetic wound treatment. As such, a better understanding of the wound characteristics or environment through in situ biomarker analysis, via for example, a smart bandage, could reduce hospitalization time, prevent amputation, aid in therapeutic studies, and improve other personalized treatments.

Further, increases in temperature over time can be linked to inflammation. Elevated levels of UA after infection can be due to upregulation of xanthine oxidase, a component of the innate immune system responding to inflammatory cytokines in chronic ulcers, which may play a role in the purine metabolism to produce UA. pH, lactate, and ammonium may all be acidity related and their elevation during bacterial infection may be monitored. Additionally, glucose levels in infected wound fluid may have a greater than approximately 35% decrease after infection, due to the increased glucose consumption of bacteria activities. The smart bandage may monitor decreases in temperature, pH, lactate, UA, and ammonium in relation to levels during infection and increases in glucose level, to indicate that treatment has improved the state of the infection in the wound. These levels may also be monitored to determine whether digestion has occurred in diabetic patients, or whether digestion is not occurring properly, as biomarkers may also change during the course of digestion.

Advances in digital health and flexible electronics have transformed conventional medicine practices into remote at-home healthcare (i.e., telemedicine). Wearable biosensors, for example a smart bandage, may allow for real-time and/or continuous monitoring of physical vital signs and physiological biomarkers in various biofluids such as sweat, saliva, and interstitial fluids, among others. Generally, wound dressings provide a moist wound environment, offer protection from secondary infections, remove wound exudate, and promote regeneration. However, chronic wounds require greater flexibility, breathability, and biocompatibility of the dressing to protect the wound bed from bacterial infiltrations and infection, and to modulate wound exudate levels. Further, chronic wounds may provide a more complex wound exudate matrix that can affect biosensor performance. As such, personalized chronic wound therapy may require close monitoring of crucial wound healing biomarkers in the wound exudate, beyond what can be discretely monitored at individual in patient visits.

To address these challenges, a fully-integrated wireless wearable bioelectronic system that more effectively monitors the physiological conditions of the wound bed via multiplexed and multimodal wound biomarker analysis, and performs combination therapy through on-demand electro-responsive controlled drug or antimicrobial agent delivery for anti-inflammatory antimicrobial treatment and exogenous electrical stimulation for tissue regeneration was developed as a smart bandage. The smart bandage may be a wearable patch that is mechanically flexible, stretchable, and can conformally adhere to the skin/wound throughout portions of, or the entirety of, the wound healing process. The smart bandage may improve comfort levels when worn by a patient and reduce skin irritation at the location the smart bandage is placed.

The smart bandage may include various biosensors that may monitor various wound biomarkers/characteristics including temperature, pH, ammonium, glucose, lactate, UA, and other biomarkers indicative of wound parameters. In various embodiments, the smart bandage may monitor, in real-time or at discrete occasions, the biomarkers or characteristics of the wound through wound exudate. The smart bandage may monitor these biomarkers or characteristics in situ using custom-engineered electrochemical biosensor arrays. The multiplexed biomarker/wound characteristic information collected by the smart bandage via the biosensors may reveal spatial and temporal changes in the wound microenvironment as well as inflammatory status of the wound through different stages of healing.

In addition to the multiplexed biosensors, the smart bandage may be equipped with an on-demand electro-responsive drug release and antimicrobial agent delivery system, loaded with an antimicrobial and/or anti-inflammatory peptide. The delivery system may release the drugs or antimicrobial agents under an applied positive voltage, such that when a positive voltage is applied, the electroactive hydrogels may release the dual-function peptide, or other drug, which can increase elimination of bacteria (or other pathogens) and modulate inflammatory responses in the wound bed during various stages of healing. In various embodiments, the on-demand delivery system may be modified with different electroactive hydrogels to deliver other drugs (including positively and negatively charged drugs and biomolecules, e.g., proteins, peptides, and growth factors). Similarly, the integration of an electrical stimulation therapeutic module may facility cell motility and proliferation, and ECM deposition and remodeling in the process of wound regeneration resulting in increased cutaneous wound healing.

In general, the combination of electrically modulated antimicrobial agent delivery and electrical stimulation on the smart bandage may accelerate chronic wound recovery and/or closure.

The embodiments described herein relate to a smart bandage for monitoring and treating wounds. The smart bandage may include a system that can monitor wound biomarkers, determine wound characteristics form the monitored wound biomarkers, perform antimicrobial agent or drug delivery, and provide electrical stimulation (ES) to the wound for healing purposes.

In various embodiments, the smart bandage may be a disposable wearable patch that includes a multimodal biosensor array for approximately simultaneous and multiplexed electrochemical sensing of wound exudate biomarkers or characteristics, a stimulus-responsive electroactive hydrogel loaded with a dual-function anti-inflammatory and antimicrobial peptide (AMP), and a voltage-modulated electrode for controlled drug or antimicrobial agent release and electrical stimulation. The biosensor array may be fabricated using microfabrication protocols on a layer of material, for example copper, followed by transfer printing onto a poly[styrene-b-(ethylene-co-butylene)-b-styrene](SEBS) thermoplastic elastomer substrate. The smart bandage may have a serpentine-like design of electronic interconnects, which, in combination with the elastic nature of SEBS, may enable increased stretchability and resilience of the smart bandage against undesirable physical deformations. The smart bandage may interface with a flexible printed circuit board (FPCB) for electrochemical sensor data acquisition, wireless communication, and programmed voltage modulation for controlled drug or antimicrobial agent delivery and electrical stimulation.

In various embodiments, the array of flexible biosensors may allow for real-time multiplexed monitoring of the wound biomarkers in complex wound exudate. The potentially continuous and selective measurement of glucose, lactate, and UA (along with other biomarkers) may be based on amperometric enzymatic electrodes with glucose oxidase, lactate oxidase, and/or uricase immobilized in a highly permeable, adhesive, and biocompatible chitosan film, respectively. In some embodiments, Prussian blue may serve as the electron-redox mediator for the enzymatic reaction that may allow the biosensors to operate at a low potential (approximately 0.0 V), which may minimize interferences of oxygen along with other electroactive molecules.

In conventional systems, due to the complex and heterogeneous composition of wound fluid (e.g., high protein levels, local and migrated cells, and exogenous factors such as bacteria), previous conventional enzymatic sensors suffered from severe matrix effects and often fail to accurately measure the target metabolite levels in untreated wound fluid. Moreover, high levels of metabolites in diabetic wound fluid, especially glucose (up to 50 mM), had posed another challenge to obtaining linear sensor response in the physiological concentration ranges. To address these issues and achieve more accurate wound fluid metabolic monitoring, the smart bandage may include a polyurethane (PU) membrane as a mass transport limiting layer that may result in enhanced linearity over a wider physiological concentration range and may increase reproducibility in complex wound fluid matrices. In some embodiments, the amperometric current signals generated from the PU-coated enzymatic glucose, lactate, and UA sensor may be proportional to the physiologically relevant concentrations of the corresponding metabolites in simulated wound fluid (SWF) with sensitivities of about 16.32, 41.44, and 189.60 nA mM⁻¹, respectively. Further, continuous monitoring of ammonium may be based on a potentiometric ion-selective electrode where the binding of ammonium with its ionophore results in an electrode potential log-linearity and may correspond to the target ion concentrations with a sensitivity of about 59.7 mV decade⁻¹. Additionally, a pH sensor may utilize an electrodeposited polyaniline film as the pH-sensitive membrane and may show a sensitivity of about 59.7 mV per pH. In further embodiments, the chemical sensors, may use a polyvinyl butyral (PVB)-coated Ag/AgCl electrode for the reference electrode, which may provide a more stable voltage that may be independent from variations in wound fluid compositions. Moreover, a gold microwire-based resistive temperature sensor may be integrated as part of the sensor array and may show a sensitivity of approximately 0.21% ° C.⁻¹ in the physiological temperature range of approximately 25-45° C.

In other embodiments, the smart bandage may perform autonomous bioanalysis, as some examples described, of wound characteristics to determine features of the wound bed, release antimicrobial agents from an antimicrobial agent reservoir, release drugs from the hydrogel, promote wound healing via exogenous electrical field stimulation, and/or communicate wirelessly with a network, personal smart devices, and/or medical equipment. In some embodiments, the smart bandage may be used to monitor and treat wounds resulting from diabetes-related illnesses. In some embodiments, the smart bandage may be used to monitor and treat wounds generally. Other types of systems are also possible, and these examples are not intended to be limiting.

Monitoring Wound Conditions and Generating Wound Treatments

Monitoring wound characteristics is an element of wound and chronic wound management. Wound characteristics generally include the characteristics and biomarkers of the physiological microenvironment of a wound. These wound characteristics can include, for example, temperature, pH, hydration, uric acid presence, ammonia, lactate, glucose, CRP concentration, biomarkers, and other physiological characteristics that may provide further insight into the condition or treatment of a wound. The presence, levels, or quantities detected of wound characteristics change at different stages of the wound healing process, and may allow for different treatments to be provided to a patient to assist with their wound recovery. For example, a wound may require antimicrobial treatment during early stages of healing and then require electrical stimulation during the final stages of healing. As described in detail below, wound healing may increase from antimicrobial or drug application in early stages, as it may reduce the infection rate of the wounds, and may benefit from electrical stimulation in later stages, as it may reduce scarring. Other benefits from treatment are also possible.

Quantitative wound characteristic observation can occur by wearable devices to alleviate or reduce the need for a patient to go to a medical facility and have a medical provider analyze or monitor the wound on a regular basis. For example, a patient may be able to extend the duration between in patient visits while still allowing a medical provider to monitor the wound through the wearable device, reducing the risk of infection and improper healing occurring without realization in between in patient visits. Moreover, wearable devices allow for wound characteristics to be monitored continuously, rather than at a snap-shot in time, which can allow for better treatment of wounds generally. Additionally, this may allow for medical providers to gain a better understanding of how the wound is healing over time, allowing for more personalized wound treatment.

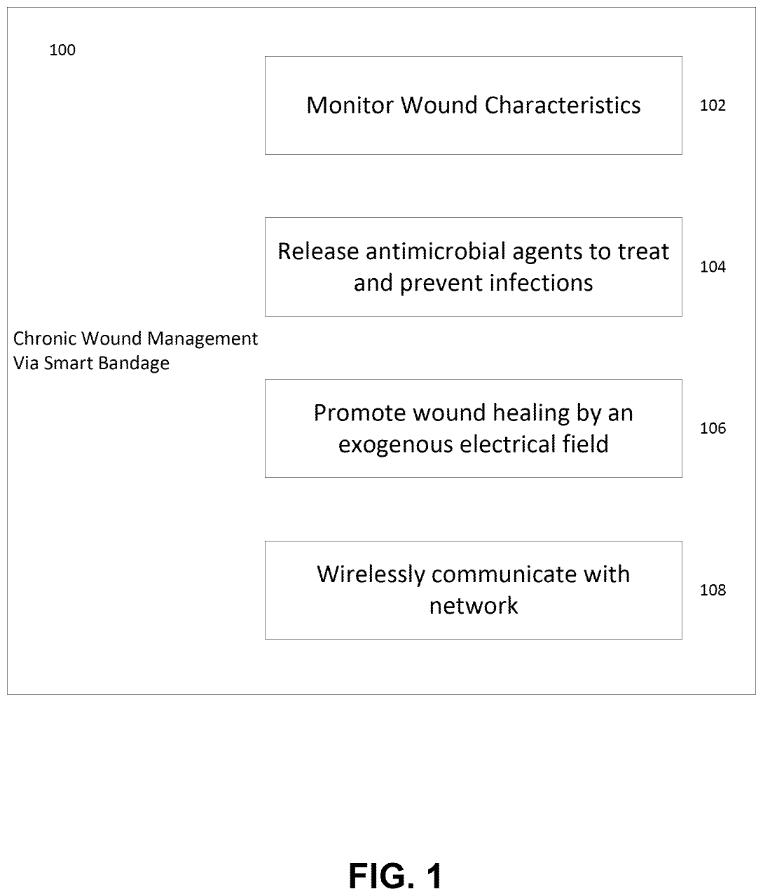

FIG. 1 an example box diagram depicting the smart bandage system 100, according to various embodiments of the disclosed technology. In some embodiments, the smart bandage system 100 may include one or more sensors that can monitor, record, and track wound characteristics. These sensors may produce signals that are representative of wound characteristics that can be transmitted to a control module, as discussed below. In some embodiments, the sensors may be biosensors. In some embodiments, a control module can perform autonomous bioanalysis of the physiological microenvironment or wound characteristics to determine a wound treatment plan 102. Wound treatment plans are based on wound characteristics, and can be determined by the patient or medical professional monitoring and treating the wound. In other embodiments, the bioanalysis performed in the control module can compare wound characteristics to threshold values and determine whether to apply treatment to the wound, if the threshold values are exceeded or otherwise met. Wound treatment may be focused on a single wound characteristic, or any combination of wound characteristics therein. Wound treatment may be directed to reducing certain values of wound characteristics. For example, a wound treatment that dispenses antibiotics may be aimed at reducing the temperature of the wound bed, which may be a bioindicator of infection.

In another example, the potential of hydrogen (pH) of the wound bed may be a characteristic that can provide information about the status of a wound during the healing process. The pH of native skin is acidic (with an approximate pH of about 4.0-6.0), while over 80% of chronic wounds with an alkaline pH (pH >8) are likely infected. This can be an indicator that is monitored by the smart bandage system 100, and reviewed by medical providers to diagnose or treat wounds at various stages of healing. In addition, the acidic environment can support proliferation of fibroblasts. In some embodiments of the present disclosure, a sensor may be located in the smart bandage that includes an individual or array of pH sensors for mapping spatial and/or temporal wound characteristics, conditions, or parameters. The pH sensor may be a pH-sensitive polyaniline film deposited on an Au electrode. In embodiments with an array of pH sensors, the smart bandage system 100 may create a map of wound characteristics. Such a map may highlight areas of greater indicators of infection and may determine a level of infection based on a given area, or overall infection levels of the wound. The map may be compared to future, past, or control maps to determine whether a wound is healing, worsening, or remaining stagnant. In some embodiments, the pH sensors may be constructed with a measuring electrode, reference electrode, a temperature sensor, a preamplifier, and an analyzer/transmitter. In some embodiments, the measuring electrode may be sensitive to the hydrogen ion, or develop a potential (voltage) directly related to the hydrogen ion concentration of a wound. In some embodiment, the pH sensor materials may be fabricated from materials such as electropolymerized polyaniline films or other carefully selected fabrication materials.

In another example, temperature of the wound may be a characteristic that can provide information regarding local blood flow and lymphocyte extravasation, as well as wound infection and chronicity. Local blood flow and lymphocyte extravasation can be a characteristic of a wound, and thus, may be helpful in determining how to treat the wound. In some embodiments of the present disclosure, a sensor may be located in the smart bandage that includes an individual or array of temperature sensors for spatial and/or temporal mapping of wound characteristics. As discussed in relation to the pH sensor, the temperature sensors may form a map of the wound bed to assist with diagnosis and treatment. In various embodiments, smart bandages that include both pH sensors and temperature sensors may allow for medical providers or other users to perform real-time adjustments and calibration of the enzymatic biosensors based on temperature and pH variations to improve realization of more accurate metabolite analysis.

In further examples, the uric acid (UA) level in a wound may be linked to wound severity and may significantly reduce during bacterial infection due to catabolysis by microbial uricase. In some embodiments of the present disclosure, a sensor may be placed in the smart bandage that includes an individual or array of UA sensors used for spatial and/or temporal mapping of mapping wound characteristics. As discussed in relation to the pH sensor, the UA sensors may form a map of the wound bed to assist with diagnosis and treatment.

In further examples, the ammonia level in a wound may be linked to the health of a cell and can be a factor when determining wound treatment, as well as, wound healing classifications. In some embodiments of the present disclosure, a sensor may be placed in the smart bandage that includes an individual or array of ammonia sensors used for spatial and/or temporal mapping of mapping wound characteristics. As discussed in relation to the pH sensor, the ammonia sensors may form a map of the wound bed to assist with diagnosis and treatment.

In further examples, the lactate level in a wound can be indicative of pre-sepsis or sepsis in a wound, as well as reflective of overall wound health. In some embodiments of the present disclosure, a sensor may be placed in the smart bandage that includes an individual or array of lactate sensors used for spatial and/or temporal mapping of wound characteristics. As discussed in relation to the pH sensor, the lactate sensors may form a map of the wound bed to assist with diagnosis and treatment.

In further examples, the CRP may be used as an inflammatory biomarker that may indicate the presence of infections when there is an increase of local concentration. In some embodiments of the present disclosure, a sensor may be placed in the smart bandage that includes an individual or array of CRP sensors used for spatial and/or temporal mapping of mapping wound characteristics. As discussed in relation to the pH sensor, the CRP sensors may form a map of the wound bed to assist with diagnosis and treatment.

In further examples, the glucose level may be used to determine a wound, for example diabetic wound, status and patient's health. In this example, variations in wound glucose levels can reflect blood glucose levels and serve as a predictive biomarker for diabetes. Further, high levels of glucose stiffen arteries and cause narrowing of blood vessels. The result being poorer levels of circulation (compared to medically accepted standard levels) near a wound that decreases the amount of blood flow and oxygen that reach the wound, which can decrease the ability for a wound to heal. Poor circulation may decrease the amount of blood flow and oxygen that reaches the wound, which may prevent or inhibit the ability for a wound to heal. In some embodiments of the present disclosure, a sensor may be placed in the smart bandage that includes an individual or array of glucose sensors for spatial and/or temporal mapping of mapping wound characteristics. As discussed in relation to the pH sensor, the glucose sensors may form a map of the wound bed to assist with diagnosis and treatment. In these examples, the glucose level of the wound may be wound characteristics, and the control module may receive signals representative of the glucose level of the wound and determine a ratio of glucose of the wound to control or standard glucose levels.

In various embodiments, the smart bandage may include pH sensors, ammonia sensors, temperature sensors, UA sensors, CRP sensors, glucose sensors, lactate sensors, and/or other biosensors that can measure the status, parameters, or characteristics of a wound. In various embodiments, the smart bandage may include any combination of the referenced sensors or other carefully selected sensors. In such examples, any combination of sensors may form a spatial or temporal map of the wound bed to assist with diagnosis, wound analysis, and wound treatment. In various embodiments, the sensors in the smart bandage may be flexible or rigid sensor patches. In other embodiments, the sensors may be biosensors. In further embodiments, the sensors may be flexible or rigid biosensors.

In some embodiments, the sensors can be prepared based on lased-engraved graphene (LEG) technology, which can provide unique electrochemical properties arising from fast electron mobility, high current density, and ultra large surface area. In this embodiment, graphene is a possible material for building high-performance sensors to detect high and low levels of UA in body fluids. In further embodiments, differential pulse voltammetry (DPV) may be used as the UA detection method herein such that the analyte level will be determined from peak current. The LEG technology may be used to fabricate any number of sensors in the smart bandage.

Further, a laser-engraved graphene (LEG) sensor may be advantageous because it may be printed using a modified conventional printer. Printable wearable sensor patches may be fabricated on a large scale at a relatively low cost. This may allow for sensor patches, which may be worn by an individual for an extended of time, for instance twelve to twenty-four hours, to be replaced regularly while limiting associated costs. Low cost printable, wearable, and disposable patches offer the opportunity to replace a patch monthly, weekly, daily, or even hourly, on a patient and collect health information over a period of several days or weeks without invasive testing and the need for a patient to come into a physical laboratory for repeated testing. Monitoring may occur both during periods of exercise and at rest. Monitoring may also occur continuously or at discrete intervals of regular or irregular timing. For example, a smart bandage may monitor wound characteristics continuously, every 5 minutes, every hour, or once a day. Other time intervals may exist as this example is not intended to be limiting.

In some embodiment, the CRP sensor may be developed from a combination of molecularly imprinted polymers (artificial antibodies) with LEG, wherein the LEG may have unique properties for designing resistive physical sensors: as temperature rises, its conductivity increases owing to increased electron-phonon scattering and thermal velocity of electrons in the sandwiched layers.

In some embodiments, the smart bandage may use the wound characteristics to determine whether the wound is healing, maintaining status, or degrading. In some embodiments, the smart bandage may be able to communicate wound characteristics to a network, wireless smart device, or medical center, continuously, systematically, or on discrete occasions, which could be set by the patient, medical provider, and/or other persons involved with wound management. For example, a smart bandage may be able to wirelessly transmit wound characteristics to a medical provider continuously, every 5 minutes, hourly, daily, or weekly. Other time intervals may exist. In other embodiments, the wound characteristics may assist in determining a wound treatment plan that may include administering antimicrobial agents via the smart bandage, and/or administering electrical stimulation (ES) via the smart bandage.

Wound Treatment Via Antimicrobial Agents

In addition to the multiplexed and multimodal biosensing, the smart bandage may be able to perform a combination of treatments for wounds through drug and antimicrobial agent release from an electroactive hydrogel layer and/or electrical stimulation under an exogenic electric field (discussed below). Both treatments may be controlled by one or more voltage-modulated electrode(s). In various embodiments, the drug delivery or antimicrobial agent release system release may include an electroactive hydrogel that may include chondroitin 4-sulfate, a sulfated glycosaminoglycan that may further include units of glucosamine, crosslinked with 1,4-butanediol diglyceryl ether. Other electroactive polymers and compositions may be included or replaced therewith. In some embodiments, the hydrogel can be fabricated using a 3D printer. Further, negatively charged CS hydrogel may be used for loading and controlling drug release/antimicrobial agent delivery because of the positively charged large biological drug molecules based on an electrically modulated ‘on/off’ drug release mechanism. For example, an AMP, TCP-25 may be loaded within the CS hydrogel network the electrostatic interactions with the polymer backbone, which may provide up to approximately 15% loading efficiency. In some embodiments, a highly porous hydrogel network under equilibrium swelling may further increase the drug loading efficiency.

Further, under an applied positive voltage, the electroactive hydrogels may rapidly protonated, which may result in anisotropic and microscopic contraction followed by syneresis/expelling of water from the hydrogel, and consequently may provide for a controlled release of the drug/antimicrobial agent, e.g., TCP-25 AMP. As discussed below. The electrical field may also help facilitate the diffusion of positively charged AMP out of the stimuli-sensitive CS hydrogel towards the cathode due to electrophoretic flow.

In some embodiments, the smart bandage may include a locally activated drug release system 104. The locally activated drug release system may include the antimicrobial agents and drugs enclosed in an antimicrobial reservoir, which may be a CS hydrogel, which may be disposed on the flexible multi-layer substrate. The antimicrobial reservoir may feed into antimicrobial outlets that dispense or release antimicrobial agents or drugs onto the skin, or into the wound, of a patient.

In some embodiments, the drugs may not be limited to antimicrobial agents, and can be in the form of anti-inflammatory drugs, growth factors, proteins, peptides, nanoparticles, microparticles.

Antimicrobial agents are compounds that kill microorganisms or inhibit their growth. Antimicrobial agents may be grouped according to the microorganisms they act upon. For example, antibiotics are generally used against bacteria, and antifungals are generally used against fungi. They can also be classified according to their function. For example, agents that kill microbes are typically microbicides, while those that merely inhibit their growth are typically called bacteriostatic agents. In some embodiments, the smart bandage may include a system that releases antimicrobial agents in any therapeutically relevant combination as described. For example, a wound with a bacterial infection may be covered with a smart bandage including antibiotics for treatment of the bacterial infection.

In various embodiments, the antimicrobial agents may be released through the antimicrobial outlets as a result of a wound treatment plan, wherein wound characteristics exceeding a specified or determined threshold may trigger the prescribed treatment. Such a treatment may include releasing antimicrobials. When wound characteristics are outside of a specified threshold, the smart bandage may be able to automatically, reactively, or directly administer antimicrobial agents to the surface or near surface of a wound.

In various embodiments, the antimicrobial agents may be released remotely, on-demand, by a patient or medical professional. When the antimicrobial agents are released remotely, they may be set to release upon a time-based occasion, specified occasion, in response to events (such as a doctor monitoring the wound), or at the patient or medical provider's discretion, i.e., on-demand.

In various embodiments, antimicrobial agents may include any form of topical or inserted agents, drugs, pharmaceuticals, and/or carefully selected therapeutic equivalents.

In various embodiments, the antimicrobial agents may be stored in an antimicrobial reservoir disposed in the flexible multi-layer substrate. In some embodiments, the antimicrobial reservoir may transfer antimicrobial agents to outlets that may dispense antimicrobial agents after a threshold wound characteristics has been reached into the patient's wound. In other embodiments, the smart bandage may dispense antimicrobial agents as a result of the patient or medical professional monitoring and/or treating the patient. In further embodiments, the smart bandage may dispense antimicrobial agents as a result of the wound treatment plan. In some embodiments, there may be a single antimicrobial reservoir disposed on the flexible multi-layer substrate. In some embodiments, there may be multiple antimicrobial reservoirs disposed on the flexible multi-layer substrate. In some embodiments, there may be antimicrobial reservoirs disposed internally within the flexible multi-layer substrate, where an outlet connects the antimicrobial reservoir to the surface of the patient's skin.

Wound Treatment Via Electrical Field

In various embodiments, the smart bandage may include an electrical stimulation (ES) module 106. ES can use an electrical current to transfer energy to a wound. The electrical stimulation produced may provide the smart bandage with an increased therapeutic capability toward enhanced tissue regeneration.

There are two general types of ES for wound care: first, is capacitively coupled electrical stimulation that involves the transfer of electric current through an applied surface electrode pad that is in wet (electrolytic) contact (capacitively coupled) with the external skin surface and/or wound bed; and second, is the application of exogenous or endogenous electrical fields (EF). Other ES treatment may exist.

In some embodiments, the smart bandage may use capacitively coupled electrical stimulation to promote wound healing. This system may work by influencing the body's own bioelectric system, such that the system can attract the cells to repair, change the cell membrane permeability, enhance cellular secretion through cell membranes, and/or orient cell structures. In some embodiments, the smart bandage may implement a capacitively coupled ES system.

In some embodiments, the smart bandage may use exogenous or endogenous EF. For example, an EF used to treat wounds utilizes a voltage or electromotive force (EMF), which may be capable of moving charged particles or ions, across cell membranes in wound tissues that lie between two electrodes applied to the body.

To produce directed current flow, there may be a source of free electrons from the ES module, conveyed to the patient via conductive electrodes that are positioned to distribute the flow of a quantity of EF energy into wound and periwound tissue. With direct current (DC) and monophasic pulsed current (MPC), the two electrodes may be polarized with regard to each other, with one being negative (cathode) and the other being positively charged (anode). Currents with polarity are used for wound healing to ostensibly replicate/activate the disturbed endogenous polarized currents that are present after wounding of the integument.

Research has shown that endogenous, measurable EFs are created by transmembrane voltages that are found in cell membranes and that when the epithelium of human skin is wounded, a low resistance pathway is created where the transepithelial potential voltage, drives current out of the wound. After wounding, in a moist wound environment, there is a lateral voltage gradient of approximately 140 mV/mm at the wound edge that decays to approximately 10 mV/mm at 500-1,000 micrometers from the wound edge. In some embodiments, exogenous EF may produce ES that can provide a directional vector as well as a non-vector activating mechanism to stimulate cells involved in wound healing by enhancing cellular motility in the wound and along an edge of the wound.

ES devices and modules can be classified according to the voltage range delivered to treatment of the wound. Low-voltage ES may have driving voltages of less than 35 volts. High-voltage ES may have driving voltages of equal to, or greater than, 35 volts. In some embodiments, the smart bandage may produce low-voltage ES. In other embodiments, the smart bandage may produce high-voltage ES. In some embodiments, the smart bandage may produce different voltages at different times, during different stages of wound healing, or in response to different wound characteristics. For example, during an initial stage of wound healing, a high-voltage may be applied to the wound bed, and then during a later stage of healing a low-voltage may be applied to the wound bed. In such an embodiment, the smart bandage may be able to apply both voltages at different times during wound healing and may be able to switch between voltages wirelessly based on a wound treatment plan or direct interference by the patient, medical provider, or similar persons.

ES may affect the biological phases of wound healing in various ways. During the inflammation phase, ES may initiate the wound repair process by its effect on the current of injury, increase blood flow, promote phagocytosis, enhance tissue oxygenation, reduce edema perhaps from reduced microvascular leakage, attract and stimulate fibroblasts and epithelial cells, stimulate DNA synthesis, control infection, solubilize blood products including necrotic tissue, and similar healing effects. During the proliferation phase, ES may stimulate fibroblasts and epithelial cells, stimulate DNA and protein synthesis, increase ATP generation, improve membrane transport, produce better collagen matric organization, stimulate wound contraction, and similar healing properties. During the epithelialization phase, ES may stimulate epidermal cell reproduction and migration, produce a smoother/thinner scar, and similar healing properties. In various embodiments, the smart bandage may be applied to a wound to produce any one or more of the above-referenced effects, along with other effects on wound healing.

ES can be an effective wound treatment plan approach for pressure ulcers stage I through IV, diabetic ulcers due to pressure/insensitivity/dysvascularity, venous ulcers, traumatic wounds, surgical wounds, ischemic ulcers, vasculitic ulcers, donor sites, wound flaps, burn wounds, and/or similar. In various embodiments, the smart bandage may be applied to a wound to treat any one or more of the above-referenced wounds, along with various other wounds.

User Communication

In some embodiments, a smart bandage may also include a wireless communication module that can wirelessly communicate with a user interface. A user interface may be available on the smart bandage, mobile device, for instance via an application, or similar devices, such as a computer in a medical provider's office. A user, may be a patient, doctor, medical professional or provider, and/or similar, and may access data collected from the smart bandage via the user interface. Data collected from the smart bandage may be transmitted such that it can be accessed via the user interface using a wireless method, such as Bluetooth, Wi-Fi, or any other one-way or two-way communication feature.

In some embodiments, the smart bandage may include a memory to store wound characteristics and wound treatment plans, and may transmit this information to a device having a user interface. The data from the smart bandage may be transmitted via Bluetooth or a similar communication method. The method may also include a further step of accessing sample data collected by a smart bandage using a user interface.

System Configuration

The smart bandage may be mass-producible and readily reconfigurable for various wound care applications. For example, in the case of chronic ulcers, the wound characteristics and microenvironment may vary from site to site, making localized monitoring important for optimized assessment and treatment of chronic wound infection. In such examples, the smart bandage may include spatial mapping of the physiological conditions of wounds during the healing process.

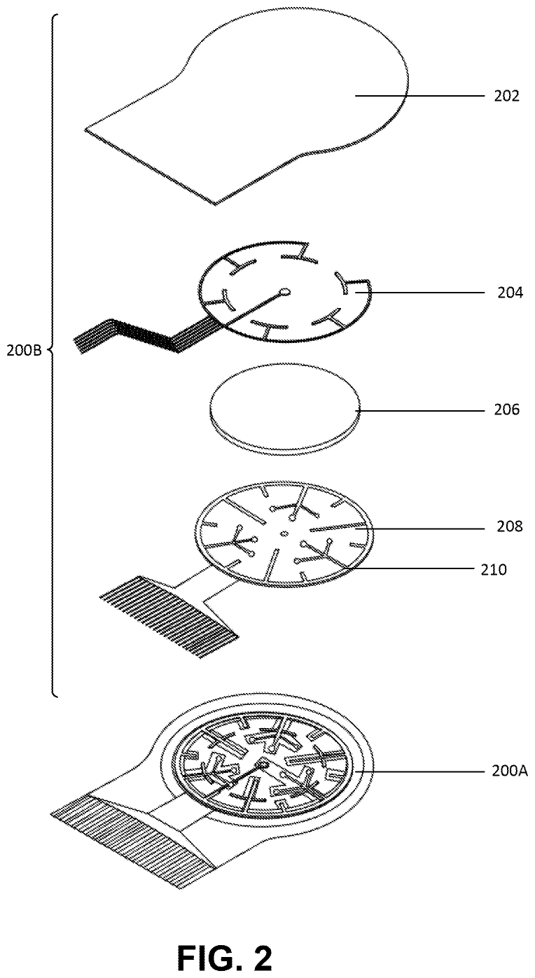

In various embodiments, the smart bandage may be constructed from a series of layers. FIG. 2 depicts an example smart bandage device 200A and an exploded example smart bandage device 200B, according to various embodiments of the disclosed technology. In some embodiments, the smart bandage 200 may include a flexible multi-layer substrate 202; one or more antimicrobial reservoirs or hydrogel layers 204 that hold antimicrobial agents or drugs 206 that can be activated to release said agents or drugs onto a patient; and an array of sensors 208.

In some embodiments, the flexible multi-layer substrate 202 may be a topical bandage made from silicone, acrylate, hydrocolloid, synthetic based rubber, medical tape, and/or similar materials that can hold the antimicrobial reservoirs 204, antimicrobial agents 206, and array of sensors 208. The flexible multi-layer substrate 202 may have a medical adhesive on one or more sides or surfaces of the substrate. The flexible multi-layer substrate 202 with the medical adhesive may hold or adhere the smart bandage 200 to the patient's skin. The flexible multi-layer substrate 202 may cover the wound partially or entirely, and in some embodiments, may be secured or adhered to the patient's skin with enough force to hold the wound together, and function as a replacement to, or in addition to, stitches, staples, or medical glue, typically used for closing a wound. In some embodiments, the flexible multi-layer substrate 202 may be stretchable, breathable, and/or carefully selected to move or remain immobile on a patient's skin. In some embodiments, the flexible multi-layer substrate 202 may encompass the antimicrobial reservoir 202, ES module, sensors, control module, and/or wireless communication module.

In embodiments, the flexible multi-layer substrate may be constructed, in part or in full, from SEBS substrate with serpentine-like electronic interconnects. Due to the soft SEBS substrate and serpentine-like design of the electronic interconnects, the smart bandage may have increased levels of mechanical flexibility and stretchability, which may afford increased levels of contact between the smart bandage and the skin of the patient. These increased levels of contact may improve the effects of the antimicrobial agent release/drug delivery and electrical stimulation via the ES module on chronic wound healing. Further, the smart bandage may stretch in various directions (e.g., unidirectionally) or incur mechanical bending without affecting sensor responses, i.e., the smart bandage may incur various physical deformations without loosing accuracy of the biosensors, as discussed below.

In some embodiments, the flexible and stretchable substrate is not limited to SEBS polymer and can include but is not limited to other flexible substrates such as poly (glycerol-co-sebacate) acrylate (PGSA), polyurethane, polydimethylsiloxane (PDMS) etc.

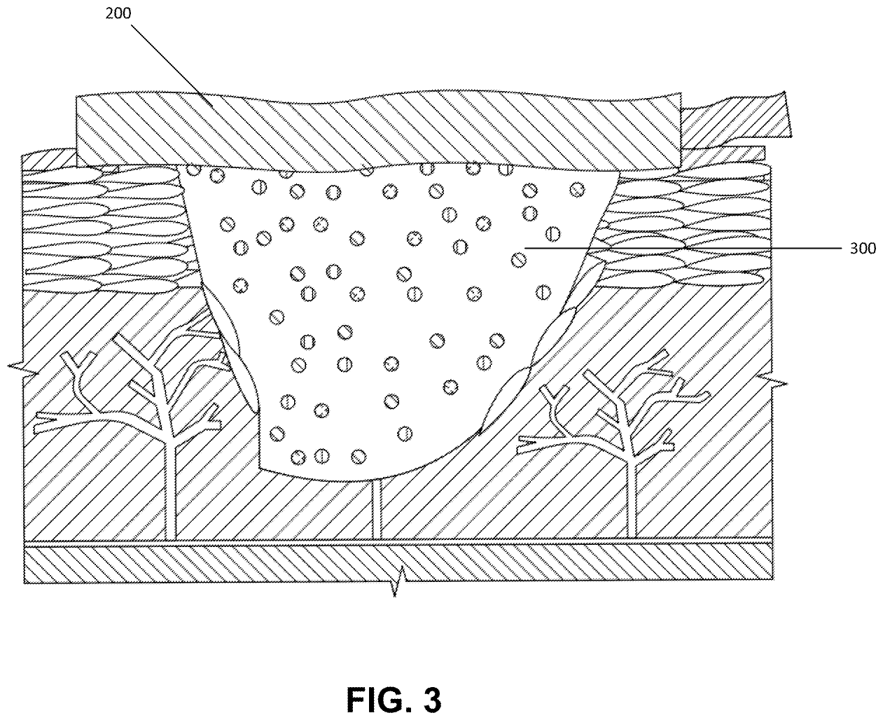

In some embodiments, the antimicrobial reservoirs or hydrogel layer 204 may be disposed on the flexible multi-layer substrate 202 and provide a system that allows for antimicrobial agents or drugs 206 to be released from the smart bandage and directly applied to the skin of a patient or into the wound on a patient, as depicted in reference to FIG. 3. The antimicrobial reservoir may open or close based on received signals from a control module, discussed below. In other embodiments, the hydrogel layer may release drugs via a positive or negative applied voltage, as discussed above. When the antimicrobial reservoir or hydrogel layer receives a signal to dispense the antimicrobial agents or drugs it may open or release agents allowing a dose of the antimicrobial agent or drug to flow through an outlet onto the patient's skin.

In further embodiments, the antimicrobial reservoirs 204 may provide a system that allows for antimicrobial agents or drugs to be administered to the interior of the wound. The antimicrobial reservoir 204 may include a chamber that contains the antimicrobial agent 206. Such a chamber may be, for example, a plastic tank with antibiotic therein. In other embodiments, the antimicrobial reservoir 204 may include multiple chambers that can hold various antimicrobial agents 206. In other embodiments, the agent may be stored in a CS hydrogel or other electroactive hydrogels. Such embodiments may allow for different antimicrobial agents or drugs 206 to be dispensed into the wound bed at various points during wound treatment. In some embodiments, the antimicrobial reservoir or hydrogel layer 204 may be locally disposed on the smart bandage, for example, on or in the flexible multi-layer substrate 202. The antimicrobial reservoir or hydrogel layer 204 may be manufactured into the flexible multi-layer substrate 202 or adhered to the flexible multi-layer substrate to allow for easier replacement and servicing of the antimicrobial reservoir 204. In other embodiments, the antimicrobial reservoir 204 may be located external to the smart bandage device 200. In such an embodiment, the antimicrobial reservoir 204 may be connected to the smart bandage device 200 by medical tubing or similar transfer mechanisms to allow for larger quantities of antimicrobial agent or drugs 206 to be stored in the antimicrobial reservoir or hydrogel layer 204 and subsequently dispensed through outlets on the smart bandage device 200.

In some embodiments, the antimicrobial reservoir or hydrogel layer 204 may carry one to fifteen doses of antimicrobial agent or drug, that, in some embodiments, can be refilled by the patient or medical provider. In other embodiments, the antimicrobial reservoir or hydrogel layer may carry more than fifteen doses of an agent. For example, in an embodiment where the dosage of an agent is low, more doses may be stored in a smaller area. In other embodiments, the antimicrobial reservoirs or hydrogel layers can be discarded and replaced with a new, full reservoir or hydrogel after each use. The antimicrobial reservoirs or hydrogel layer 202 can also be replaced with a different reservoir or hydrogel that has a different antimicrobial agent or replaced with a reservoir containing other medications, such as pain relievers or antibiotics. As such, the antimicrobial reservoir or hydrogel layer may be replaced with a reservoir containing the same or similar antimicrobial agent, or containing an entirely different agent altogether. In some embodiments, there may be multiple antimicrobial reservoirs or hydrogel layers on the smart bandage, and the respective antimicrobial agents can be administered alone/individually, or in any combination thereof.

In various embodiments, the smart bandage device 200 may include a various sensors 208. The sensors 208 may be biosensors. Biosensors may be configured to detect a wide variety of organic compounds present in a wound bed. For example, metabolites, amino acids, vitamins, minerals, hormones, antibodies, pH, UA level, ammonia level, lactate level, CRP level, glucose level and other compounds may be detected. The biosensor may be a sodium sensor. The biosensor 108 may be other sensors such as enzyme sensors, impendence sensors, tissue-based sensors, antibody sensors, DNA sensors, optical sensors, electrochemical biosensors, piezoelectric sensors, bacteria sensors, and/or similar biosensors. A smart bandage device 200 may also include a temperature sensor. A temperature reading, in conjunction with detected concentrations of key organic compounds, may provide an indication of health or wound status or characteristics. Additionally, a temperature measurement over time, along with correlated measurements of concentrations of key organic compounds, may provide indications about changing wound status or may reveal fluctuations indicative of infection or other health conditions that would not be revealed by a one-time test, such as a blood test or in patient wound monitoring. An electrolyte reading may indicate a patient's hydration status and/or electrolyte balance. As with a temperature measurement, an electrolyte measurement, especially over a continuous period and in conjunction with other measurements, may reveal changing wound status, fluctuations indicative of infection, or a particular health condition.

The biosensors may be arranged in various sensor arrays that may include any number of sensors. For example, a sensor array may include seven pH sensors and nine temperature sensors for monitoring and mapping size and thickness of infected chronic wounds. The spatial and temporal mappings of wound beds may be constructed from any number of sensor values monitoring during the wound healing process, and may further indicate that a wound has improved. For example, more uniform sensor readings across a wound bed may indicate that the wound has improved in relation to the prior readings, which may have included areas with increased sensor reading indicative of infection.

In various embodiments the biosensors arrays may include any type of sensors and are not limited to pH and temperature sensors.

In various embodiments, the smart bandage device 200 may include a control module. The control module may receive signals from the sensors 202 representative of characteristics of the wound and sends signals to the antimicrobial outlet to dispense antimicrobial agent and the ES module to produce electrical stimulation. In particular, the control module may interface with a flexible printed circuit board (FPCB) for electrochemical sensor data acquisition, wireless communication, and programmed voltage modulation for controlled drug or antimicrobial agent delivery and electrical stimulation. The control module may locally determine to administer antimicrobial agents and ES, or may be wirelessly directed to administer antimicrobial agents and ES. In embodiments where the control module operates locally or autonomously, threshold wound characteristic values may be used to determine whether to administer treatment. For example, the control module may be programed to administer antibiotics if the control sensor receives signals from the temperature sensor that indicate the wound bed is over 38 degrees C. (as increased wound bed temperature may be a bioindicator of bacterial infections). Threshold wound characteristic values, such as temperature, may be preprogrammed into the control module, or may be dynamically added by medical providers or patients in response to varying conditions of the wound. Threshold wound characteristic values may be values programmed into the control module that, when surpassed or met, initiate the control module to send signals to the antimicrobial reservoir, hydrogel layer, or ES module to administer treatment.

In other embodiments, the control module may receive signals from the sensors representative of wound characteristics and transmit those signals via a wireless communication module to a mobile device, computer, and/or network. Once the signals are read by the patient or medical provider, the patient or medical provider may transmit signals back to the control module on the smart bandage device 200 representative of treatment methods. For example, the temperature sensor may send signals representative of the wound bed exceeding 38 degrees C. to a medical provider's computer, the medical provider may read the signals (in the form of an application that processes the signals into a more easily readable form), and transmit signals back to the control module representative of a prescribed treatment method, i.e., releasing antibiotics.

The control module may include instructions executable on a non-transitory computer readable storage medium that can process the received signals from the sensors and wireless communication module and transmit signals to the antimicrobial reservoir, hydrogel layer, and ES module or wireless communication module.

As discussed above, the ES module 210 may provide electrical stimulation to a wound bed. The ES module 210 may be controlled by the control module on the smart bandage device 200. The control module may receive signals wirelessly from a mobile device, or any other one-way or two-way communication device linked or wirelessly connected to the smart bandage device 200. The signals received by the control module may be representative of levels of electrical stimulation to be provided to the wound bed, may indicate a start/stop function for providing electrical stimulation, or may provide instructions in a non-transitory computer readable storage medium that control the ES module 210. The control module and the ES module 210 may be powered by a battery (not shown). The battery may be a lithium-ion battery, or other power storage devices. The battery may be single use, or rechargeable. The battery may be recharged from movement of the smart bandage device 200. For example, the smart bandage device 200 may be placed on a patient's wrist and move when the patient moves their arm, providing kinetic energy to be transferred to the battery and stored for powering the smart bandage device 200. The system may be battery free or self-powered. For example, the system may be powered by solar energy, biofuel cells, nanogenerators, etc. The battery may also provide the power for the ES module 210 and may include voltage and current converters that can change the output voltage and current of the ES module 210. The voltage and current may be changed via instructions received or sent by the control module.

FIG. 3 is an example smart bandage 200 application to an open wound 300, according to various embodiments of the disclosed technology. In some embodiments, the smart bandage 200 may be applied to an open wound 300 to monitor the characteristics of the wound, such as pH, temperature, UA level, ammonia level, lactate level, CRP level, glucose level, and/or similar. In some embodiments, the smart bandage may be applied to closed wounds.

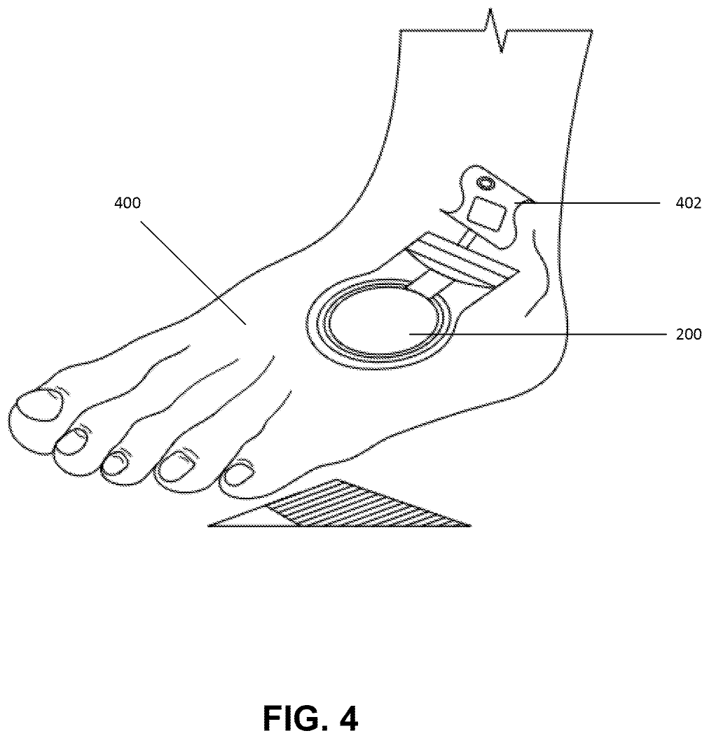

FIG. 4 depicts an example application of a smart bandage device 200 to a human foot 400, according to various embodiments of the disclosed technology. In some embodiments, the smart bandage device 200 may be placed on the skin of a patient 400, and then measure the wound characteristics statically, continuously, discretely at intervals, or in any combination thereof. As discussed, the smart bandage device 200 may be connected to additional modules 402, which may include ES stimulation modules, batteries, wireless communication modules, logic circuits (for example, circuits and modules that store instructions for wound treatment), and other modules that may control or monitor the sensors and/or treatment features of the smart bandage device 200.

By having a smart bandage device 200 placed on a patient 400, a wound may be continuously or statically monitored without the patient being in a medical facility. The result of monitoring the wound characteristics may allow for more accurate and more carefully timed and dynamic administration of antimicrobial agents, drugs, or ES, and may further enhance the accuracy of wound diagnosis. In other embodiments, the smart bandage may transmit the monitoring information to a network, computer, phone, and/or similar, where a third-party, such as a patient or medical provider, may direct the smart bandage to administer antimicrobial agents or ES.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A smart bandage device, comprising:
a substrate with multiple layers;
a sensor disposed within a first layer of the substrate, wherein the sensor measures characteristics of a wound;
a reservoir disposed within a second layer of the substrate;
an outlet disposed within the substrate adjacent to a skin-facing layer of the substrate, wherein the outlet dispenses agents from the reservoir;
an electrical stimulation module disposed within a third layer of the substrate, wherein the electrical stimulation module produces electrical stimulation to the wound; and
a control module that receives signals from the sensor representative of characteristics of the wound and sends signals to the outlet to dispense agents and the electrical stimulation module to produce electrical stimulation.

2. The smart bandage device of claim 1, further comprising a wireless communication module that connects the smart bandage device to another wireless device.

3. The smart bandage device of claim 1, wherein the sensor is a biosensor configured to detect at least one of metabolites, amino acids, vitamins, minerals, hormones, antibodies, pH, UA level, ammonia level, lactate level, CRP level, and glucose level.

4. The smart bandage device of claim 2, further comprising a non-transitory computer readable storage medium that includes instructions to:
receive signals from the sensor representative of characteristics of the wound; and
transmit the received signals via the wireless communication module to an external device.

5. The smart bandage device of claim 2, further comprising a non-transitory computer readable storage medium that includes instructions to:
receive signals from an external device via the wireless communication module; and
transmit signals to the outlet and electrical stimulation module representative of a wound treatment plan, wherein the wound treatment plan comprises a distribution of agent and an electrical stimulation.

6. The smart bandage device of claim 1, wherein the reservoir is a hydrogel layer.

7. The smart bandage device of claim 6, wherein the flexible multi-layer substrate is a topical bandage.

8. A smart bandage device for monitoring and treating wounds, comprising:
a flexible multi-layer substrate;
a plurality of sensors disposed within the flexible multi-layer substrate, wherein the plurality of sensors measure wound characteristics and produce signals reflective of the wound characteristics;
a control module that receives the signals from the sensors and performs a bioanalysis of the wound characteristics, wherein the control module generates a wound treatment plan;
an antimicrobial reservoir disposed on an external surface of the flexible multi-layer substrate, wherein the control module sends signals to the antimicrobial reservoir to release antimicrobial agents out of antimicrobial agent outlets when threshold wound characteristics are met;
an electrical stimulation module disposed within the flexible multi-layer substrate, wherein the electrical stimulation module produces electrical stimulation; and
a wireless communication module disposed within the flexible multi-layer substrate that connects the smart bandage to a wireless device.

9. The smart bandage device of claim 8, wherein the plurality of sensors are configured to detect at least one of metabolites, amino acids, vitamins, minerals, hormones, antibodies, pH, UA level, ammonia level, lactate level, CRP level, and glucose level.

10. The smart bandage device of claim 8, wherein the control module further comprises a non-transitory computer readable storage medium that includes instructions to:
receive signals from the plurality of sensors representative of characteristics of the wound; and
transmit signals to the antimicrobial reservoir when threshold wound characteristics are met.

11. The smart bandage device of claim 8, wherein the control module further comprises a non-transitory computer readable storage medium that includes instructions to:
receive signals from the plurality of sensors representative of characteristics of the wound; and
transmit signals to the electrical stimulation module when threshold wound characteristics are met.

12. The smart bandage device of claim 8, wherein the antimicrobial reservoir is a hydrogel layer.

13. A method of monitoring and treating a wound using a smart bandage, comprising:
applying a smart bandage to a wound on a patient, wherein the smart bandage comprises:
a substrate with multiple layers;
a sensor disposed within a first layer of the substrate, wherein the sensor measures characteristics of a wound;
an antimicrobial reservoir disposed within a second layer of the substrate;
an antimicrobial outlet disposed within the substrate adjacent to a skin-facing layer of the substrate, wherein the antimicrobial outlet dispenses antimicrobial agents from the antimicrobial reservoir;
an electrical stimulation module disposed within a third layer of the substrate, wherein the electrical stimulation module produces electrical stimulation to a patient; and
a control module that receives signals from the sensor representative of characteristics of the wound and sends signals to the antimicrobial outlet to dispense antimicrobial agents and the electrical stimulation module to produce electrical stimulation;
receiving signals from the sensors representative of wound characteristics, wherein the received signals are transmitted to the control module;
comparing the received signals representative of wound characteristics to threshold wound characteristics values; and
applying a treatment to the wound when the threshold wound characteristics values are exceeded by the received signals representative of wound characteristics.

14. The method of claim 13, wherein applying treatment further comprises administering an agent to the wound on the patient.

15. The method of claim 13, wherein applying treatment further comprises administering an electrical stimulation to the wound on the patient.

16. The method of claim 13, wherein the threshold wound characteristics values are programmed into the control module.

17. The method of claim 16, wherein the threshold wound characteristics are dynamically adjusted by a medical provider during wound treatment.

18. The method of claim 13, wherein receiving signals from the sensors representative of wound characteristics comprises receiving signals from a sensor configured to measure at least one of metabolites, amino acids, vitamins, minerals, hormones, antibodies, pH, UA level, ammonia level, lactate level, CRP level, and glucose level.

19. The method of claim 13, wherein applying treatment to the wound when the threshold wound characteristics values are exceeded is done autonomously.

20. The method of claim 13, further comprising wirelessly communicating with another wireless device.
